SEQUENCE LISTING (7 sequences)

SEQ ID NO: 1 - DNA, 23 bp, Artificial Sequence
  PAM sequence
gtcaccaatcctgtccctagtgg

SEQ ID NO: 2 - RNA, 100 nt, Artificial Sequence
  sgRNA guide sequence
gucaccaauccugucccuagguuuuagagcuagaaauagcaaguuaaaauaaggcuaguccguuaucaacuugaaaaaguggcaccgagucggugcuuuu

SEQ ID NO: 3 - DNA, 23 bp, Artificial Sequence
  PAM sequence 2
gctgaagcactgcacgccgtagg

SEQ ID NO: 4 - DNA, 140 bp, Artificial Sequence
  HDR template
accctgaagttcatctgcaccaccggcaagctgcccgtgccctggcccaccctcgtgaccaccctgagccacggggtgcagtgcttcagccgctaccccgaccacatgaagcagcacgacttcttcaagtccgccatgcc

SEQ ID NO: 5 - DNA, 720 bp, Artificial Sequence
  EGFP sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 6 - RNA, 96 nt, Artificial Sequence
  sgRNA guide sequence 2
gcugaagcacugcacgccguguuuuagagcuagaaauagcaaguuaaaauaaggcuaguccguuaucaacuugaaaaaguggcaccgagucggugc

SEQ ID NO: 7 - DNA, 9177 bp, Artificial Sequence
  Cas9/sgRNA plasmid sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctgaagcactgcacgccgtgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2022/0333129 A1
(43) Pub. Date: Oct. 20, 2022

(54) A NUCLEIC ACID DELIVERY VECTOR COMPRISING A CIRCULAR SINGLE STRANDED POLYNUCLEOTIDE

(71) Applicant: LightBio Limited, Hampton (GB)

(72) Inventors: Thomas Adie, Hampton (GB); Paul Rothwell, Hampton (GB); Maria Barreira Gonzalez, Hampton (GB)

(21) Appl. No.: 17/754,203

(22) PCT Filed: Sep. 28, 2020
(86) PCT No.: PCT/GB2020/052341

§ 371 Date: Mar. 25, 2022

(30) Foreign Application Priority Data
Sep. 26, 2019 (GB) 1913898.1

(51) Int. Cl. C12N 15/85; C12N 15/113

(52) U.S. Cl. CPC C12N 15/85; C12N 15/113; C12N 2310/532

(57) ABSTRACT

The invention relates to a delivery vector for the delivery of a single-stranded nucleic acid. Said vector is a closed circular polynucleotide comprised of at least three sections, two of which have sufficient complementarity to form a duplex, and an intervening sequence containing the single-stranded nucleic acid to be delivered. Said duplex includes a recognition sequence for a targeted nuclease such that under appropriate conditions the single-stranded nucleic acid is released.

### FIELD OF THE INVENTION

This invention relates to the intracellular delivery or release of single stranded nucleic acid, in particular a single stranded donor oligonucleotide. Whilst any type of nucleic acid is contemplated here, single stranded deoxyribonucleic acid (DNA) may be preferred.

### BACKGROUND OF THE INVENTION

The most flexible regions of nucleic acids are often non-base paired and include single stranded deoxyribonucleic acid (ssDNA) and ribonucleic acid (ssRNA) regions that are involved in vital processes within the cell.

Single stranded nucleic acid molecules are of interest to those skilled in the art of delivering nucleic acid to cells in particular, since the nucleic acid is immediately available within the transfected cell, and does not require “unwinding” by an appropriate enzyme to expose ssDNA sections, for example. These sections are then available for transcription into ssRNA, such as messenger RNA (mRNA), or for interaction with other proteins that recognise the ssDNA.

Single-stranded DNA (ssDNA) is an essential intermediate in many biological processes that include replication, recombination, repair, transcription, and transposition of DNA. As such, it is also desirable to introduce ssDNA into cells in order to have a therapeutic effect exploiting these mechanisms, amongst others. ssDNA can also have many therapeutic uses. In a eukaryotic cell, ssDNA is frequently exposed as a result of many cellular processes, including replication, transcription, and recombination.

The exposed ssDNA is vulnerable to chemical attack and nucleolytic degradation; therefore, it must be properly protected to avoid mutations. Single-stranded DNA binding proteins (SSBs) immediately bind to ssDNA to protect it from inappropriate reactions until the relevant process is complete. However, ssDNA not so protected would be vulnerable to chemical attack and nucleolytic degradation.

Single stranded nucleic acid molecules are of interest to those skilled in the art of delivering nucleic acid to cells in particular, since the nucleic acid is immediately available within the transfected cell, and does not require “unwinding” by an appropriate enzyme to expose the relevant genetic information (for example for transcription and translation or insertion into the genome). They are considered to be an optimal delivery vector for several applications, not least gene transfer, gene editing and bio-sensing.

Alternatively, the single stranded nucleic acid may have a function related to its conformation, i.e. as an aptamer or nucleic acid enzyme (including DNAzymes and RNAzymes).

Further, the provision of single stranded nucleic acid in a cell may be required in antisense applications. Antisense therapy involves the use of oligonucleotides with a particular sequence which is complementary to a target sequence, such as messenger RNA (mRNA).

Even further, the provision of a single stranded nucleic acid in the cell may be required for translation purposes, i.e. to provide mRNA to directly instruct protein production within the cell. Therefore the single stranded nucleic acid may be an RNA, particularly an mRNA. Other types of RNA may also be delivered using the vector, including long noncoding RNA (lncRNA), microRNA (miRNA), Piwi-interacting RNA (piRNA), small interfering RNA (siRNA), short hairpin RNA (shRNA), trans-acting siRNA (tasiRNA), repeat associated siRNA, enhancer RNA, antisense RNA, guide RNA, small nucleolar RNA or small nuclear RNA.

Both ssDNA and dsDNA donor sequences can act as efficient gene-editing templates. ssDNA donor templates have been found to have a unique advantage in terms of repair specificity when used in gene editing (Design and specificity of long ssDNA donors for CRISPR-based knock-in; Han Li, Kyle A. Beckman, Veronica Pessino, Bo Huang, Jonathan S. Weissman, Manuel D. Leonetti bioRxiv 178905), and therefore their use is desirable.

Despite it being desirable to provide single stranded nucleic acid to cells, there may be some drawbacks in this approach. The efficacy of nucleic acid therapies can be limited by unwanted degradation. Chemical modifications are known to improve nucleic acid stability, but the type, position, and numbers of modifications all make a difference, and it may be desirable to minimise the number of modifications in some instances, since the use of modifications can affect the ability of the nucleic acid to bind to proteins.

By its very nature, single stranded nucleic acid is quickly degraded within cells, since free 3′ and 5′ ends are available for enzymatic degradation, such as by the action of nucleases, which “chew back” the ends and destroy the nucleic acid. For example, Trex1, the major 3′ to 5′ DNA exonuclease in mammalian cells, acts preferentially on single-stranded DNA (ssDNA), to which it binds avidly. Mutations in the human TREX1 gene can cause Aicardi-Goutières syndrome, characterized by perturbed immunity. Modification of the nucleotides at the 3′ end has been shown to assist resistance to such degradation.

It has been appreciated for some years that cytosolic DNA is immune stimulatory, particularly the innate immune system. DNA is normally present in the nucleus of eukaryotic cells, and the presence of DNA in atypical locations, including the cytoplasm and endosomes, is understood to trigger immune activation. The presence of DNA anywhere other than the nucleus is thought to trigger DNA recognition systems to detect both DNA genomes of invading pathogens and incorrectly working cells.

Unmethylated CpG DNA motifs, which are abundant in the genomes of many pathogens, are known to be able to stimulate immune responses. Single-stranded DNAs with specific signatures, including AT-rich stem loop regions are also known to activate immune responses.

Therefore, it is desirable to deliver single stranded nucleic acid in a “covert” way, by delivering the linear single stranded nucleic acid in a vector that is not as easily recognised by the innate immunity systems, and then releasing the linear single strand within the cell.

The present inventors have devised a delivery vector that enables the release of a single strand of nucleic acid under appropriate conditions, such as in the nucleus or other compartment of a target cell. The single stranded nucleic acid may be delivered for any conceivable purpose wherein a linear single stranded nucleic acid is desirable, for example as a donor for gene editing, for translation in the cell, for modifying gene expression, for provision of entities such as aptamers and nucleic acid enzymes or for antisense applications.

### SUMMARY OF THE INVENTION

The present invention relates to a delivery vector, for the delivery of a linear single stranded nucleic acid to a cell, preferably a target cell. The delivery vector is effectively a carrier or delivery vehicle into which the single stranded nucleic acid is placed. Processes within the cell can exploit the structure of the delivery vehicle and release the single stranded nucleic acid. The construct can be manipulated or designed such that the release of the single stranded nucleic acid occurs only under particular conditions or cell types.

The provision of a delivery vector for a linear single stranded nucleic acid permits the covert delivery of an entity that is otherwise quickly degraded. The delivery can be controlled or directed.

According to one aspect, the present invention provides:

a nucleic acid delivery vector comprising a circular single stranded polynucleotide said vector comprising:

(a) a duplex formed from a first section and a third section of said polynucleotide, said sections including sequences which are complementary;

(b) a loop formed from a second section, said section separating the first and third sections;

wherein said duplex includes a recognition sequence for a targeted nuclease.

The nucleic acid delivery vector is described as a single stranded polynucleotide, since under denaturing conditions, the delivery vector is a closed circular polynucleotide.

The delivery vector preferably delivers a linear single stranded nucleic acid. Said single stranded nucleic acid is present within the second section of the delivery vector and the sequence of the second section and the linear single stranded nucleic acid are thus substantially the same. The linear single stranded nucleic acid may take any appropriate conformation and be of any appropriate sequence. The linear single stranded nucleic acid may be a nucleic acid enzyme, an aptamer, a donor template or an antisense nucleic acid, or any of the single stranded nucleic acids discussed herein. The linear single stranded nucleic acid has a free 5′ and 3′ end, once released from the delivery vector. Depending on the nature of the nuclease, the single stranded nucleic acid may be released with fragments of the first and third sections still present. This is the case for nucleases such as Cas9, and is depicted in FIGS. 1A and 1B. These fragments may be small, preferably less than 15 bases in length.

The targeted nuclease may be any nuclease that is target specific. The duplex of the delivery vector includes a recognition site for a targeted nuclease. This nuclease may recognise this sequence independently or it may require the help of a guide sequence. The action of the nuclease on the recognition site is the cleavage of the duplex, or at least a strand thereof. Said cleavage may be a blunt cleavage or a staggered cut. The nuclease can be endogenous or exogenous, and if it is the latter, this is also supplied to the cell. The nuclease can be delivered to the cell separately, or it can be delivered by including the mRNA sequence or DNA gene sequence for the nuclease in the delivery vehicle. Should the latter delivery vector be supplied, in effect it provides its own nuclease for releasing the single stranded nucleic acid. The nuclease is preferably an endonuclease. The nuclease may be any entity that can appropriately cleave a phosphodiester bond in a sequence specific manner.

The delivery vector may be used in a cell. The delivery vector may be used to provide a donor template for genome editing. The delivery vector may be used to deliver antisense nucleic acid to a cell. The delivery vector may be used to deliver a nucleic acid enzyme or aptamer to a cell. The delivery vector can be used to deliver any single stranded RNA or nucleic acid hybrid to the cell.

As used herein the cell may be a mammalian cell, preferably a human cell and preferably a human somatic cell.

According to another aspect, the present invention provides a method of providing a linear single stranded nucleic acid to a cell, comprising the use of a delivery vector as herein described.

According to another aspect, the present invention provides a method of providing a linear single stranded donor oligonucleotide or template to a cell for genome editing, comprising the use of a delivery vector as described herein.

With any of the methods described herein, the delivery vector of the invention is introduced into a cell. The delivery vector can be transfected by any suitable means, including chemical, physical or viral.

### DETAILED DESCRIPTION OF THE INVENTION

The construct of the invention is a closed circular polynucleotide comprised of a single strand. The sequence of said construct is designed such that it can be apportioned into at least three sections, although the boundaries of these sections may be adaptable, varying depending on the cleavage site of the nuclease. The construct is preferably a delivery vector, intended for the delivery of a linear single stranded nucleic acid. The first section and third sections include some complementarity, such that under appropriate or physiological conditions, these sections are capable of base-pairing and forming a duplex or stem structure. This duplex is a result of self-complementary sequences within the polynucleotide. The second section intervenes between the first and third sections, and includes the sequence for the linear single stranded nucleic acid for delivery. This second section, since it is positioned between the two complementary sequences, will generally form a “loop” of nucleic acid, which starts and ends at the first and third sections which are duplexed. It will generally be represented as a single strand, since no regions of complementarity with other sequences within the polynucleotide will intentionally be included in order for the delivery vector to work as intended. However, single stranded nucleic acids are well known for assuming some secondary structure, and therefore this second section may be in any possible conformation, including one or more of hairpins, loops, and pseudoknots, including sections of linear nucleic acid. The “loop” as used herein may also be considered to be the section of the polynucleotide that is not designed to form the duplex part of the delivery vector, but this does not preclude it from self-annealing. If homology arms are present, these may be capable of self-annealing into a further duplex. The loop is effectively the “single stranded” sequence for delivery, trapped by the duplex section which can be processed in order to release the single strand. Whilst the term “loop” is used, it is clear that larger loops (for example, 6 nucleotides and above) may have their own secondary structure.

For simplicity, the delivery vector is represented in the figures as a closed circular polynucleotide which includes a duplex section and a looped out single stranded section. Those skilled in the art will appreciate that this structure is entirely simplified and that the looped out section may include one or more secondary structures. The attached FIG. 1A depicts a delivery vector where the duplex is a stem, and the rest of the polynucleotide loops out from one end of the stem. The “single stranded” or second section of the delivery vector thus is captured or held in place by the duplex, said duplex being formed by the first and third sections. It will be appreciated that although there is a loop formed between the duplex due to the presence of the second section of the delivery vector, this loop may have its own secondary structure, and therefore not appear as a single stranded loop upon inspection. The sequence of the loop (and thus the second section) may preferably be designed to have little if any complementarity to the first and third sections of the polynucleotide. A polynucleotide is a polymer whose molecule is composed of many nucleotide units. The polynucleotide (delivery vector) may be any suitable length, for example from 50 or 100 residues/nucleotides/bases to 10,000 residues/nucleotides/bases. Thus, the polynucleotide may be from 100, 200, 300, 400, 500, 600, 700, 800, 900 or 1000 residues/nucleotides/bases. The polynucleotide may be up to 10,000, 9,000, 8,000, 7,000, 6,000, 5,000, 4,000, 3,000, 2,000 or 1,000 residues/nucleotides/bases. The polynucleotide may be any number of residues/nucleotides/bases between these values.

The duplex section may be any suitable length, to accommodate the recognition sequence for a nuclease. As a minimum, the duplex may be at least 4 base pairs in length, but may be, as a minimum, 5, 6, 7, 8, 9 or 10 base pairs in length. The duplex may indeed be longer and may be up to 100, 250, 500, 750 or 1000 base pairs in length in some instances. Sections of complementary sequence may surround the nuclease recognition sequence in the duplex to assist duplex formation.

The polynucleotide may be composed of any nucleotides. These nucleotides may be natural, modified or artificial. The nucleotides may be polymerised to form RNA, DNA, locked nucleic acid (LNA), peptide nucleic acid (PNA), morpholino nucleic acid, glycol nucleic acid (GNA), threose nucleic acid (TNA), hybrids and mixtures thereof and any other artificial (xeno) nucleic acids. It may be preferred that the polynucleotide is DNA or a modified version thereof (i.e. with modifications in the backbone, sugar residue or nucleobase). If modifications are used, these will be such that binding or activity as required is not impaired. Those skilled in the art are aware of how to determine whether modifications affect binding capacity or activity using standard assays.

The polynucleotide is circular or contiguous. There are, therefore, no free 3′ or 5′ ends present in the polynucleotide.

The polynucleotide includes the sequence for a single stranded linear nucleic acid which it is desired to deliver to a cell.

The polynucleotide is effectively a delivery vehicle or vector for a linear single stranded nucleic acid which does have free 3′ and 5′ ends. This linear single stranded nucleic acid may be of any appropriate length and any sequence. Due to the propensity of single stranded nucleic acid to form secondary structures, although the single stranded nucleic acid is depicted as a long chain of nucleotides, it is likely that self-complementary regions will anneal, resulting in secondary structures such as hairpins, stems, loops, pseudoknots and cruciforms.

The linear single stranded nucleic acid for delivery may be for any intended purpose.

Thus, the linear single stranded nucleic acid may be an antisense molecule, which is designed with a sequence that base pairs with a complementary RNA strand, in order to prevent said RNA strand working in the usual way, for example to be translated into a protein if the RNA is messenger RNA (mRNA).

The linear single stranded nucleic acid may be an RNA with a function in the cell, such as mRNA, long noncoding RNA (lncRNA), microRNA (miRNA), Piwi-interacting RNA (piRNA), small interfering RNA (siRNA), short hairpin RNA (shRNA), trans-acting siRNA (tasiRNA), repeat associated siRNA, enhancer RNA, antisense RNA, guide RNA, small nucleolar RNA or small nuclear RNA.

The linear single stranded nucleic acid may comprise a sequence that forms a secondary structure that has a function, such as a nucleic acid enzyme or aptamer.

The linear single stranded nucleic acid may be a donor template for genome editing. This is described in more detail below.

The linear single stranded nucleic acid is the part of the polynucleotide designated “second section” herein. It is effectively restrained within the delivery vector until the conditions are such that the nucleic acid may be released.

The sequence of the polynucleotide may be designed to include the first, second and third sections described herein. The sequence, or order of the nucleotides, can be designed such that there are self-complementary sequences to enable the duplex to be formed. These self-complementary sequences are clearly on the same strand of polynucleotide, and therefore the base pairing is only possible if the sections align with one section in the 5′ to 3′ direction and the other section in the 3′ to 5′ direction.

Those skilled in the art will appreciate that this naturally occurs in single stranded polynucleotide molecules.

Complementarity is achieved by distinct interactions between nucleobases: adenine (A), thymine (T) (uracil (U) in RNA), guanine (G) and cytosine (C). Where reference is made to complementary sequences and the like, this refers to the nucleotide base-pairing interaction of one nucleic acid sequence with another nucleic acid sequence that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which the sections anneal to complementary or substantially complementary sections are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349, 1968. In general, whether such annealing or binding takes place is influenced by, among other things, the length of the section and their complementary section, the pH, the temperature, the presence of mono- and divalent cations, the proportion of G and C nucleotides in the sequences, the viscosity of the medium, and the presence of denaturants. Such variables influence the time required for base pairing or annealing. Thus, the preferred conditions will depend upon the particular application. Such conditions, however, can be routinely determined by persons of ordinary skill in the art, without undue experimentation. Typically, conditions are selected to allow complementary or substantially complementary sections to selectively bind or anneal with their corresponding section, but not hybridize to any significant degree to other sequences in the polynucleotide. Thus, appropriate conditions are selected such that the self-complementary sequences present in sections one and three are capable of annealing and forming a duplex. It is ideal that no other sequences are included in the polynucleotide that are capable of annealing to either of these sections, to avoid unwanted duplex formation.

The sequences of the first and third sections of the polynucleotide are designed or created such that they form a duplex under the appropriate conditions discussed above. For use in human or animal therapy, the duplex will form under physiological conditions. Those skilled in the art will be able to determine what physiological conditions are. These are, in general, conditions of the external or internal milieu that may occur in nature for that organism or cell system, in contrast to artificial laboratory conditions. A temperature range of 20-40 degrees Celsius, atmospheric pressure of 1, pH of 6-8, glucose concentration of 1-20 mM, atmospheric oxygen concentration, earth gravity and electromagnetism are examples of physiological conditions for most earth organisms.

The stability of this duplex is determined by its length, the number of mismatches or bulges it contains (a small number may be tolerable, especially in a long duplex) and the base composition of the two regions. Pairings between guanine and cytosine have three hydrogen bonds and are more stable compared to adenine-thymine/uracil pairings, which have only two. Base stacking interactions, which align the pi bonds of the bases' aromatic rings in a favourable orientation, also promote duplex formation.

The duplex is an important processing site for the delivery vector. The duplex provides a section of “double stranded” nucleic acid, whilst the secondary structure of the remaining part of the polynucleotide forming the delivery vector is more variable. This duplex, shown on the Figures as a stem, could equally be any secondary structure including a hairpin (where the first and third sections are therefore contiguous), stem loop (where a fourth section intervenes between the first and third sections) or even part of a more complex structure such as a pseudoknot, or cruciform. All that is relevant to the invention at hand is that there is a duplex present.

The sequence of the duplex is designed or created such that the duplex includes a recognition sequence for a targeted nuclease, preferably an endonuclease. Alternatively put, the duplex provides a site which can be targeted for cleavage by an appropriate entity. Thus, the duplex provides not only a means of capturing or restraining the linear single stranded nucleic acid for delivery, but also the means to release or deliver said linear single stranded nucleic acid as and when appropriate. The duplex therefore includes a target sequence (which may also be designated a recognition sequence) and a cleavage site designed to allow the release of the linear single stranded nucleic acid.

The cleavage site may allow for a blunt cleavage of the duplex, or a staggered cut of the duplex with a strand overhang. The cleavage site may allow for only one of the strands to be cleaved. Preferably both strands are cleaved. The cleavage of the duplex will be determined by the type of nuclease used. It will be understood by those skilled in the art that, depending on the nature of the cleavage site, fragments of the duplex may remain present at the 3′ and 5′ ends of the single stranded nucleic acid molecule. Indeed such fragments are represented in FIGS. 1A and 1B (see 7a and 7b). Such fragments may be advantageous, in that they provide an immediate “buffer” portion that may be degraded by the nucleases in the cell.

The recognition sequence for a targeted nuclease may be any desirable sequence, depending on the targeted nuclease.

A targeted nuclease is a nuclease, such as an endonuclease (an enzyme that cleaves the phosphodiester bond within a polynucleotide chain), that recognises a particular sequence. This recognition is either inherent (i.e. they recognise a particular target or recognition sequence by itself) or this recognition is guided by a separate entity (for example a guide nucleic acid). A recognition sequence for either an inherent or guided nuclease can be used in the delivery vector of the invention.

Nucleases may have an inherent ability to recognise a specific sequence; said specific sequence is herein termed a recognition sequence. In some embodiments, the recognition sequence will be a palindromic sequence about four to six nucleotides long. Most nucleases cleave the duplex unevenly, leaving complementary single-stranded ends. There are hundreds of nucleases known, each using a different recognition sequence. Endonucleases are divided into three categories, Type I, Type II, and

Type III, according to their mechanism of action. Those skilled in the art will be capable of identifying suitable nucleases and the appropriate recognition sequence that needs to be included into the duplex in order to allow for specific cleavage to occur.

Targeted nucleases, or programmable site-specific nucleases, include zinc-finger nucleases (ZFNs), transcription activator-like effector nucleases (TALENs), and meganucleases (MNs). Targeting these nucleases to specific sequences requires protein engineering, but those skilled in the art will be aware of the requirements for protein engineering in order to produce a nuclease to recognise and cleave a particular sequence. Such nucleases are in use for gene editing.

Nucleases may also be guided to the recognition sequence. The “guide” in this instance may be any suitable nucleic acid molecule or derivative thereof, such as DNA or RNA or a hybrid thereof. Thus, the nuclease may be a RNA guided nuclease or a DNA guided nuclease. As the technology evolves, it is likely that nucleases will be discovered that can be guided with artificial nucleic acid molecules too. Thus, in this embodiment, the recognition sequence is complementary or substantially complementary to a guide nucleic acid, said guide nucleic acid which recruits the nuclease to the correct site of action.

Alternatively, the delivery vector itself can provide one or more self-cleaving nucleases or other entities capable of cleavage within the duplex in a sequence specific manner.

Currently, the most well-known guided nuclease is Cas9 (CRISPR associated protein 9). Cas9 is an RNA-guided DNA nuclease enzyme associated with the CRISPR (Clustered Regularly Interspaced Short Palindromic Repeats) adaptive immunity system in Streptococcus pyogenes. As used in nature, Cas9 interrogates and cleaves foreign DNA, such as from invading bacteriophage or bacterial plasmid. Cas9 performs this interrogation by checking for sites complementary to the 20 base pair spacer region of the guide RNA. If the DNA substrate is complementary to the guide RNA, Cas9 cleaves the invading DNA. Cas9 has gained a great deal of interest in recent years because it can cleave nearly any sequence complementary to the guide RNA. Because the target specificity of Cas9 stems from the guide RNA:DNA complementarity, engineering Cas9 to target new DNA is straightforward, simply by designing an appropriate guide. The programmable sequence specificity of Cas9 has been harnessed for genome editing and gene expression control in many organisms. Native Cas9 requires a guide RNA composed of two disparate RNAs that associate—the CRISPR RNA (crRNA), and the trans-activating crRNA (tracrRNA). Cas9 targeting has however been simplified through the engineering of a chimeric single guide RNA (sgRNA) or hybrid DNA/RNA guide. The Cas9 protein remains inactive in the absence of guide RNA. In engineered CRISPR systems, guide sequence is comprised of a single strand of RNA or RNA/DNA that may form a T-shape comprised of one tetraloop and two or three stem loops. The guide sequence is engineered to have a 5′ end that is complementary to the target DNA sequence, which is generally around 20 bases in length. For Cas9, guide sequences have been shown to work in the range of 17 to 24 bases in length.

The guide RNA or RNA/DNA binds to complementary target DNA. Directly adjacent to the target DNA sequence is protospacer adjacent motif (PAM), this is a 2-6 base pair DNA sequence immediately following the DNA sequence targeted by the Cas9 nuclease. It is generally thought that Cas9 will not successfully bind to or cleave the target DNA sequence if it is not followed by the PAM sequence. The canonical PAM is the sequence 5′-NGG-3′ where “N” is any nucleobase followed by two guanine (“G”) nucleobases, and this is associated with the Cas9 nuclease of Streptococcus pyogenes (SpCas9), whereas different PAMs are associated with the Cas9 orthologues from other bacteria. 5′-NGA-3′ can be a highly efficient non-canonical PAM for human cells. Attempts have been made to engineer Cas9s to recognize different PAMs, and therefore it is just a matter of matching the engineered Cas9 to the relevant PAM.

Thus, when designing a duplex for targeting by Cas9 or similar nuclease, consideration of inclusion of a target sequence and a PAM sequence (if the target is DNA) is required in order to provide a viable recognition and cleavage site.

Cas9 is subject to modification and mutagenesis, and engineered Cas9 variants are available. A definition of variants is included below.

Work is underway to identify other guided nucleases, particularly those that can be used in CRISPR systems.

Some have already been identified. These include:

Cas12a: Clustered Regularly Interspaced Short Palindromic Repeats from Prevotella and Francisella 1 or CRISPR/Cpf1 (also known as Cas12a): Cpf1 is an RNA-guided nuclease of a class II CRISPR/Cas system. Cpf1 genes are associated with the CRISPR locus, coding for an nuclease that use a guide RNA to find and cleave viral DNA. Cpf1 is a smaller and simpler nuclease than Cas9, overcoming some of the CRISPR/Cas9 system limitations. This enzyme may recognize a T-rich PAM, and create a staggered, double-stranded DNA cut with a 5′ overhang.

Cas13a (from the bacterium Leptotrichia shahii) is an RNA-guided enzyme system that targets RNA rather than DNA, but does not seem to require a PAM; instead what may be relevant is the Protospacer Flanking Site (PFS).

Thus, the duplex formed in the polynucleotide includes the recognition sequence for a targeted nuclease. This recognition sequence can be designed to match the recognition sequence for a nuclease that can recognise sequences inherently (i.e. by itself) or matches the guide sequence for a guided nuclease. Since in the latter case the guide sequence may also be designed by the skilled user, this gives the greatest flexibility to choose a sequence to ensure that the sequence is not common or found in the cell of interest, such as in the genome of the cell undergoing editing, for example. Should a guided nuclease be used, the recognition sequence for a targeted nuclease may also need to include a PAM or PFS, as appropriate.

The delivery vector of the present invention may have utility in methods of genome editing, or gene editing in cells, particularly eukaryotic cells, notably mammalian cells, including human cells.

The delivery vector of the present invention may be used to deliver a single stranded nucleic acid to a cell, particularly a eukaryotic cell, notably a mammalian cell, including a human cell.

Should it be desirable to undertake genome or gene editing of a cell, those skilled in the art understand that there are several elements that must be provided to the cell. The first is a sequence specific nuclease in order to introduce a double-strand break (DSB) in the DNA at a predetermined point. The genome or gene editing then utilises the machinery of the cell in repairing that break, primarily by providing to the cell an exogenous donor nucleic acid with which to repair the DSB. Thus, the machinery to introduce the cleavage site is introduced, along with the donor sequence needed to repair it.

Homology-directed repair (HDR) is a process of homologous recombination where a template is used to provide the homology necessary for precise repair of a double-strand break (DSB). This is one of the natural mechanisms utilised for genome or gene editing.

Currently, exogenous repair (or donor) templates can be delivered into a cell, most often in the form of a synthetic, single-strand DNA donor oligo or DNA donor plasmid. However, as discussed previously, there are issues in delivering single stranded nucleic acids into the cell. There is also the issue that the single stranded nucleic acid may be delivered off-target to other cells where no genome or gene editing is required.

The delivery vector of the present invention has a significant advantage over the use of single stranded linear donor templates. The delivery vector may be designed such that the same nuclease is used to target both the genome and the delivery vector, thus providing a two-step system to ensure that the single stranded template is only released in cells in which the correct machinery for genome or gene editing has been included. Thus, should the delivery vector be used for delivering a single stranded nucleic acid donor templates, it can take advantage of the same technology to release the donor template ready for use and to introduce the DSB in the genome required for the editing, thus piggybacking upon the system already delivered to the target cell.

The genome editing may be in any part of the genome. The term ‘genome’ generally refers to the entire sequence of DNA of an organism. The genome includes genes: a gene is a sequence of nucleotides in DNA or RNA that codes for a molecule that has a function. The genome also includes regions of DNA that promote or inhibit gene activity, and regions that do not appear to affect protein production or function. Any one or more of these may be edited, as required. Gene editing may be a primary use, since the direct editing of genes may have therapeutic implications for numerous diseases and conditions.

Should the delivery vector be used for genome editing, the second section of the polynucleotide is a sequence suitable for use as a nucleic acid donor template. RNA or DNA templates are possible, but DNA is currently more preferred. Thus, the polynucleotide is preferably a DNA. Should the single stranded donor template require the use of HDR in order to effect the gene editing, it is usual to include homology arms within the donor template. HDR relies on the presence of a donor template with sufficient homology to the regions flanking the cut site, these are the homology arms.

Important parameters for consideration in the success of genome editing include homology arm length and homology arm symmetry. It is generally accepted that using current technologies, that homology arm lengths of 30, 40 and 50 nucleotides has optimal efficiency, while longer lengths such as 50 or 60 nucleotides have a lower efficiency. Whether the homology arms should be symmetrical (i.e. the same length) depends on the nature of the donor template. For unmodified single stranded donor templates, asymmetrical homology arms have a better efficiency, but if phosphorothioate modifications are made, symmetrical homology arms appear to be more effective. Those skilled in the art of genome editing are aware of the optimisation of these parameters for the particular circumstances under which they are operating.

The second section of the polynucleotide may therefore include sequences which are designated as homology arms, flanking the insert sequence. The insert sequence may be a single nucleotide for a single nucleotide substitution. The insert sequence may be an entire gene or non-coding region of the genome and may be many hundreds of nucleotide in length. Since the donor template is delivered via the delivery vector, it may be possible to include more nucleotides in the insert sequence than previously achieved with a single stranded donor template. The insert sequence may therefore be from 0 nucleotides in length to 1000 nucleotides in length. If the insert sequence is 0, this can be used to knock out single nucleotides or larger sequences in the target genome.

Use of single-strand donor template for HDR has been shown to be more efficient than using other types of donor template. Such templates can be delivered into a cell to insert or change short sequences (SNPs, amino acid substitutions, epitope tags, etc.) of DNA in the endogenous genomic target region. The benefits of using a synthetic donor template is that no cloning is required to generate the donor template and modifications can be added during synthesis for different applications, such as increased resistance to nucleases. The donor template can indeed include no nucleotides for insertion. In this embodiment, the gene or nucleotides are removed from the genome instead. The insert sequence may be any required sequence, ranging from a single nucleotide to correct a SNP to the entire sequence of a gene or genomic region. Since the insert sequence is entirely synthetic, this allows for changes in sequence to effect a change in function, if required. Thus the insert sequence can be any desired sequence, as required to perform the genome editing. It will be appreciated by those skilled in the art of genome editing that the targeted nucleases are not endogenous to the potential target cells, and therefore these must be provided to the cell in any appropriate way (genetically or directly), together with any associated guide sequences. The delivery vector of the present invention may be supplied to the cell at the same time or separately. Any suitable transfection technique may be used. The cell for transfection may be isolated, or ex vivo, for re-implantation into an organism, or the cell for transfection may be in vivo. The nuclease and any guide sequence may be provided on the same vector or different vectors. The vector may be any appropriate nucleic acid, including plasmids. Possible transfection routes include Lipofection, Electroporation, Nucleofection, Microinjection or use of a Virus. The route taken will depend upon the cell chosen for transfection and the nature of the vector used to express the nuclease and any guide sequence.

The delivery vector of the present invention may have utility in providing an antisense single stranded nucleic acid to the cell.

The delivery vector of the present invention may have utility in providing a nucleic acid enzyme or aptamer to the cell.

Where the delivery vector is not being used for genome editing, the duplex sequence may be designed with a recognition sequence for an endogenous nuclease, such that all that is required to be provided to the cell is the delivery vector of the present invention. In other embodiments, the nuclease is provided to the cell, as discussed above in relation to genome editing.

The delivery vector of the present invention may be provided to any cell including cell lines, primary cells, stem cells and the like. Somatic cells are preferred. The cell may be from any organism. The delivery vector may be transfected by any suitable means, including electroporation, lipofection, nucleofection or microinjection. Viral gene delivery methods may also be considered. The cells may be transfected ex vivo, in vitro, or in vivo.

The delivery vector and therefore the polynucleotide is preferably a synthetic molecule, and as such is manufactured in a cell-free manner. Many techniques for synthesizing a long single strand of nucleic acid of a particular sequence are known, including enzymatic amplification of a template nucleic acid, ab-initio synthesis, use of overlapping template, rolling circle amplification and the like. Strand-stripping of a double stranded polynucleotide may be necessary for techniques such as PCR, which result in a duplex.

Once a long single strand of polynucleotide has been obtained as desired, a single strand ligase may be used to seal the end. This results in a single stranded circular polynucleotide. Examples of suitable ligase enzymes include CircLigase™ from Epicentre, US. Chemical cyclisation reactions may also be employed. In the latter techniques, cyclic oligonucleotides containing a single triazole, amide or phosphoramidate analogue of the nucleic acid backbone are employed. The linear precursor can therefore include a 5′ azide and a 3′-alkyne for example, and a 1,4 triazole linkage included chemically, resulting in a circular polynucleotide.

It may be necessary to use a solid support during the preparation of the delivery vector, either in isolating the single stranded polynucleotide or in assisting the annealing of the free ends during circularisation. Binding affinity pairs such as biotin-streptavidin can be utilised with the sold support, such as on beads. The polynucleotide, for example, can be biotinylated, and streptavidin present on the bead.

Those skilled in the art will appreciate that there are numerous synthetic techniques that can be used to synthesise the polynucleotide with the desired sequence.

Variant Polypeptides:

A variant polypeptide comprises (or consists of) sequence which has at least 40% identity to the native protein. A variant sequence may be at least 55%, 65%, 70%, 75%, 80%, 85%, 90% and more preferably at least 95%, 97% or 99% homologous to a particular region of the native protein over at least 20, preferably at least 30, for instance at least 40, 60, 100, 200, 300, 400 or more contiguous amino acids, or even over the entire sequence of the variant. Alternatively, the variant sequence may be at least 55%, 65%, 70%, 75%, 80%, 85%, 90% and more preferably at least 95%, 97% or 99% homologous to full-length native protein. Typically the variant sequence differs from the relevant region of the native protein by at least, or less than, 2, 5, 10, 20, 40, 50 or 60 mutations (each of which can be substitutions, insertions or deletions). A variant sequence used in the process of the invention comprises a sequence having at least 80% identity with the native protein.

Variants of the native protein also include truncations. Any truncation may be used so long as the variant is still able to cleave a target sequence as described above. Truncations will typically be made to remove sequences that are non-essential for catalytic activity and/or do not affect conformation of the folded protein, in particular folding of the active site. Truncations may also be selected to improve solubility of the nuclease polypeptide. Appropriate truncations can routinely be identified by systematic truncation of sequences of varying length from the N- or C-terminus.

Variants of the native protein further include mutants which have one or more, for example, 2, 3, 4, 5 to 10, 10 to 20, 20 to 40 or more, amino acid insertions, substitutions or deletions with respect to a particular region of the native protein. Deletions and insertions are made preferably outside of the catalytic domain. Insertions are typically made at the N- or C-terminal ends of a sequence derived from the native protein, for example for the purposes of recombinant expression. Substitutions are also typically made in regions that are non-essential for catalytic activity and/or do not affect conformation of the folded protein. Such substitutions may be made to improve solubility or other characteristics of the enzyme. Although not generally preferred, substitutions may also be made in the active site or in the second sphere, i.e. residues which affect or contact the position or orientation of one or more of the amino acids in the active site. These substitutions may be made to improve catalytic properties.

Substitutions preferably introduce one or more conservative changes, which replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid.

It is particularly preferred that the variant is able to cleave nucleic acid as described above with an efficiency that is comparable to, or the same as the native protein, or indeed is improved with respect to speed, efficiency, accuracy or delivery.

### FIGURES

FIG. 1A is a depiction of an exemplary delivery vector of the present invention. Shown are the first and third sections (1 and 3) forming a duplex, with a recognition sequence for a nuclease depicted (4). The second section (2), herein representing a donor template for gene editing, can include homology arms (5a and 5b) and a target sequence (6).

FIG. 1B is a representation of the same delivery vector as FIG. 1A whereas the nuclease has cleaved at the target site, and the single stranded nucleic acid is released (10). It can be seen here that fragments of the first and third sections (7a and 7b) remain in the single stranded nucleic acid. These may or may not be present, depending on the nature of the nuclease and the way in which the duplex is designed.

FIG. 2 is a representation of the recruitment of a nuclease, such as Cas9 (12), to the duplex (4) using a guide sequence (gRNA in this case—11). Shown are the two cleavage sites (13a and 13b), one for each strand of the duplex.

FIG. 3 is a simplified representation of gene editing using the single stranded nucleic acid released from the delivery vector of the invention, labelled as FIG. 1A. Gene editing in this instance is via HDR. The homology arms (5a and 5b) play an important role in aligning the insert (6) for inclusion into the genome (20) which has already had a DSB introduced. The genome with the inserted sequence is also shown (21).

FIG. 4 shows a photograph of a gel prepared according to Example 1; split into three sections. Gel electrophoresis is the standard lab procedure for separating nucleic acids by size (e.g., length in base pairs) for visualization and purification. Electrophoresis uses an electrical field to move the negatively charged nucleic acids through an agarose gel matrix toward a positive electrode. Shorter DNA fragments migrate through the gel more quickly than longer ones. Thus, you can determine the approximate length of a DNA fragment by running it on an agarose gel alongside a DNA ladder (a collection of DNA fragments of known lengths). However, circular nucleic acids run differently in a gel. In section 1 the preparation of the delivery vector of the invention is applied to the gel. The delivery vector is highlighted with an arrow. The other nucleic acids present in the preparation are raw materials or side products. Section 2 includes a marker ladder (M) and depicts the application of the preparation applied in Section 1 when treated with an exonuclease—the delivery vector is immune since there are no free ends, but other fragments are degraded. The delivery vector here was designed to include a duplex to which a guide RNA would recruit Cas9. Section 3 includes a marker ladder (M).

The lane marked 3 relates to the preparation once the guide RNA and Cas9 has been introduced. The arrow here indicates the single stranded nucleic acid has been released by the action of Cas9, and thus the circular structure has been opened.

FIG. 5 is a delivery vector map showing the oligonucleotide for the vector created and used in Example 2. Shown are the sequences for the GFP gRNA and PAM, GFP to BFP single stranded oligonucleotide and the various restriction sites. The oligonucleotide is 254 base pairs in length. It can be seen that the gRNA and PAM sequences are present in the sense and antisense arrangements to allow loopback and annealing. The hairpin sequence is also shown.

FIG. 6 is the data generated from Example 2. HEK293T-EGFP cells expressing Cas9 are losing EGFP. Histograms showing GFP signal as percentage of maximum counted events as measured by flow cytometry in cells transfected with either high (450 ng) or low (45 ng) BFP delivery vector (“mbDNA”) at indicated time-points post-transfection. Dotted line indicates threshold for GFP-positive signal. Percentage of GFP-negative events in each sample are quoted. These are plots of GFP expression versus percentage of maximum count. Three sets of data are presented, the first column being the data for High BFP delivery vector (mbDNA), the middle column for low BFP delivery vector (mbDNA) and the last column for no Cas9. Results are shown for 2, 3, 5, 6 and 10 days post transfection.

FIG. 7 is the data generated from Example 2. The delivery vector (mbDNA) causes Cas9-mediated EGFP to BFP conversion in HEK293T-EGFP cells. Mean blue fluorescence of lysed cells is measured at indicated time-points post-transfection, and are plotted as raw intensities (Non-normalised) or relative to the no mbDNA control (Normalised). Data for two biological replicates are shown. Results are shown for 2, 3, 5, and 6 days post transfection).

The invention will now be demonstrated in the following examples, which are not limiting of the scope of the invention:

### EXAMPLES

### Example 1

Demonstration of Processability by Cas9

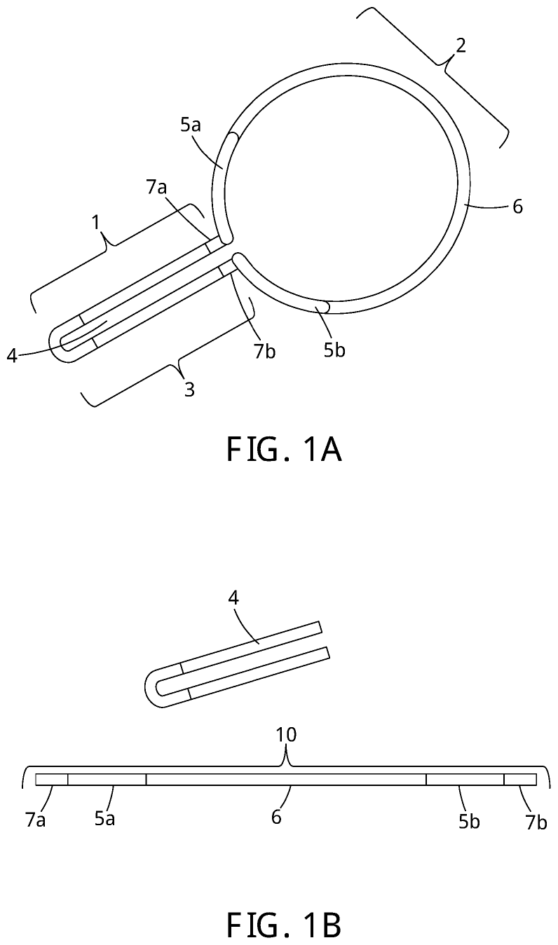

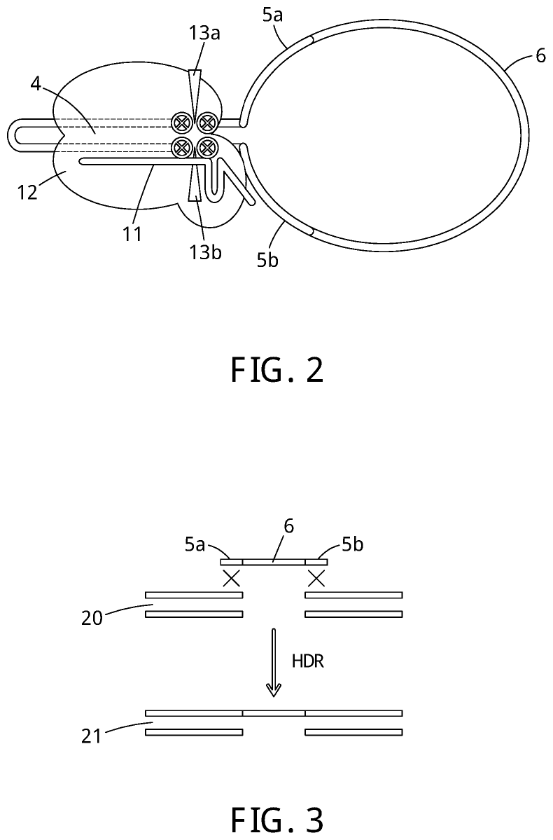

An example of the delivery vector of the invention was designed to include a duplex to which a guide RNA would recruit Cas9. The vector was produced in house as ssDNA, and ligated to seal it into the conformation depicted in FIG. 1A. A sample (sample 1) was taken. The vector was then incubated with 10 units of T5 exonuclease (NEB) at 37° C. for 3 hours. Another sample (sample 2) was taken.

A guide RNA was designed to target the duplex region of the vector and ordered, along with purified Cas9 protein, from GenScript. The sgRNA was annealed: 19.5 μl H₂O, 3 μl Cas9 reaction buffer @10×, 7.5 μl sgRNA @100 μM was combined and heated to 75° C. then left to cool to room temperature. Ribonucleoprotein was then prepared according to GenScript's direction; 0.3 μl annealed sgRNA, 0.5 μl Cas9 protein, 4 μl Cas9 reaction buffer @10×, 27.2 μl H₂O were combined and incubated at 37° C. for 10 minutes.

900 ng of the vector DNA was then added, the volume brought to 40 μl with H₂O, and the reaction incubated at 37° C. for 3 hours. A final sample was taken (sample 3).

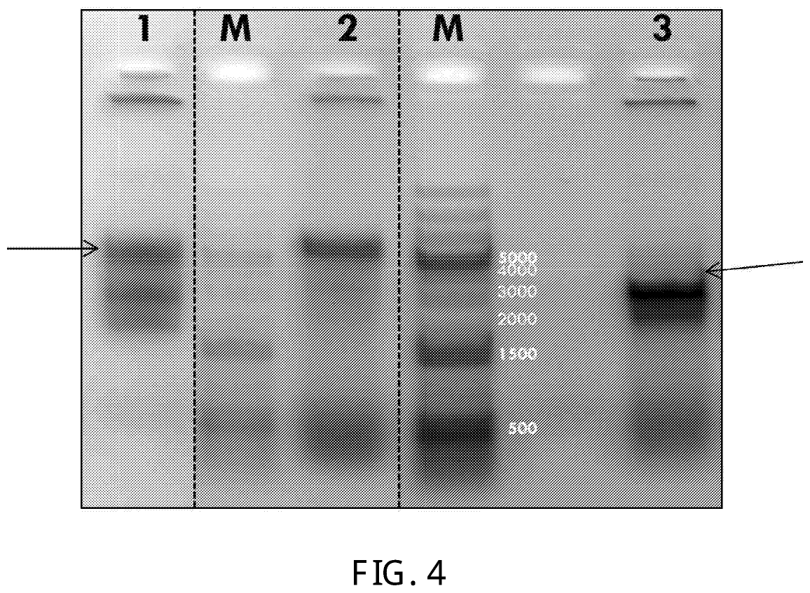

Samples 1, 2 and 3 were loaded on a 0.8% agarose TBE gel (FIG. 4: Section1: Sample 1, Section 2: Sample 2 and Section 3: Sample 3) stained with SafeView. A marker, GeneRuler 1 kb+ DNA ladder (ThermoFisher) was also loaded and the gel run to resolve the bands.

Sample 1 showed bands consistent with closed vector (indicated by the arrow on FIG. 4, section 1, and side products (unclosed vector; smaller products of the construction reaction). Sample 2 showed a strong band for the closed vector, indicating its resistance to the exonuclease, while the open vector and side product bands were reduced to a smear at the bottom of the gel. Sample 3 showed the band of the vector being successfully cleaved by Cas9—the open linear portion can now run faster and further on the gel compared to when constrained in the uncut circular form (shown with an arrow on FIG. 4, section 3).

Sequences:

In the delivery vector, the target site for the nuclease and the PAM sequence in the duplex is:

(SEQ ID No. 1)
GTCACCAATCCTGTCCCTAGTGG

The sgRNA guide sequence is:

(SEQ ID No. 2)
gucaccaauccugucccuagGUUUUAGAGCUAGAAAUAGCAAGUUAAAAU
AAGGCUAGUCCGUUAUCAACUUGAAAAAGUGGCACCGAGUCGGUGCUUUU

### Example 2

Nucleic Acid Vector Preparation

Sequences:

In the delivery vector, the target site for the nuclease and the PAM sequence in the duplex is:

(SEQ ID No. 3)
GCTGAAGCACTGCACGCCGTAGG

In the delivery vector, the sequence for the HDR template (with edited bases in lowercase and underlined) is:

(SEQ ID NO. 4)
ACCCTGAAGTTCATCTGCACCACCGGCAAGCTGCCCGTGCCCTGGCCCAC
CCTCGTGACCACCCTGAgCcACGGgGTGCAGTGCTTCAGCCGCTACCCCG
ACCACATGAAGCAGCACGACTTCTTCAAGTCCGCCATGCC

The sequence of EGFP on the genome (with bases to be edited in lowercase and underlined) is:

(SEQ ID NO. 5)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGAcCtA
CGGcGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAG

The sgRNA guide sequence is:

(SEQ ID No. 6)
gcugaagcacugcacgccguGUUUUAGAGCUAGAAAUAGCAAGUUAAAAU
AAGGCUAGUCCGUUAUCAACUUGAAAAAGUGGCACCGAGUCGGUGC

SEQ ID No. 7 is the sequence of the Cas9/sgRNA plasmid (not shown here).

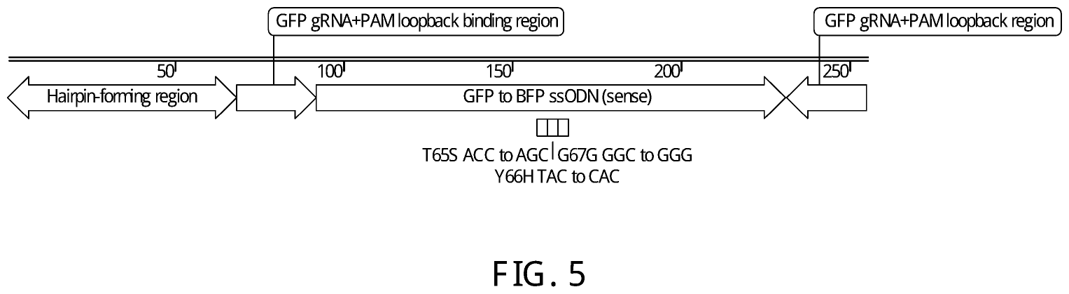

The delivery vector as depicted in FIG. 5 with a stem designed to target EGFP (EGFP is green fluorescent protein, derived from Aequorea victoria) and edit it to BFP (blue fluorescent protein) (“BFP mbDNA”) and a control vector oligonucleotide lacking the target sequence were ordered from Biolegio (Nijmegen, the Netherlands). The oligonucleotides were allowed to loopback and anneal to form a ‘lasso’-like structure in the following reaction:
8 μl oligo (100 μM) (7.5 μg/μl)
9μl ddH₂O
   Incubated for 10 minutes at 98° C., and then cooled at a rate of 0.06° C. per second to 16° C.

The annealed oligonucleotide was ligated to seal the nick in the vector backbone:
17 μl annealed vector
2 μl N buffer (10×)
   300 mM Tris pH 8.9
   300 mM (NH₄)₂SO₄
   5 mM MgSO₄
2 μl ATP (10 mM) (NEB, Ipswich, US)
1 μl T4 DNA ligase (400,000 U/ml) (NEB, Ipswich, US)
   Incubated for 7 hours at 16° C.

To remove any non-ligated single-stranded DNA, the reactions were subjected to digestion with T5 exonuclease:
20 μl ligated vector
2 μl N buffer (10×—as above)
2 μl T5 exonuclease (10,000 U/ml) (NEB, Ipswich, US)
16 μl ddH₂O
   Incubated for 12 hours at 37° C.

Annealed, ligated and T5-digested vectors were column purified using a PCR purification kit (Macherey-Nagel, Dueren, Germany).

Demonstrating Cas9 Gene Editing Ability

A HEK293T cell line stably expressing a single copy of EGFP (HEK293T-EGFP) was acquired (kind gift from Astrid Glaser). Conversion of EGFP to a blue fluorescent variant (BFP) by way of Cas9-mediated gene editing has previously been demonstrated in this cell line using single stranded oligo DNA nucleotides (ssODN) as the template (Glaser et al, Molecular Therapy, Nucleic Acids, 5(7), e334, incorporated here by reference).

DNA was delivered into either HEK293T or HEK293T-EGFP cells seeded in 6-well plates and grown in 1.5 ml complete medium (DMEM+10% FBS+2 mM glutamine) via chemical transfection using PElpro (Polyplus-Transfection®) following the manufacturer's instructions. 1.13 μg of total DNA and 3.39 μl of PElpro per transfection in a total volume of 200 μl serum-free DMEM (4.5 g/I glucose) were used. 100 ng of TIVA-pUC EF1α-Scarlet-I plasmid DNA per reaction was used to monitor transfection efficiency. In Cas9 reactions, 250 ng of Cas9+sgRNA plasmid was added. Either 450 ng (high) or 45 ng (low) of BFP or control mbDNA were used (as indicated in figures). The reactions were brought up to 1.13 μg of DNA using a blank plasmid. All transfections were performed in duplicate.

Cells were grown for indicated time periods before they were harvested via trypsinisation. Transfection efficiency (% red fluorescence) and loss of GFP intensity over time was monitored on a CytoFLEX flow cytometer (Beckman Coulter, High Wycombe, UK). Cells were lysed with RIPA buffer to release their protein contents. Relative blue fluorescence intensity of lysed cells (protein) across samples was measured using a Spark® microplate reader (Tecan, Männedorf, Switzerland) with excitation at 360 nm and emission at 465 nm.

Results:

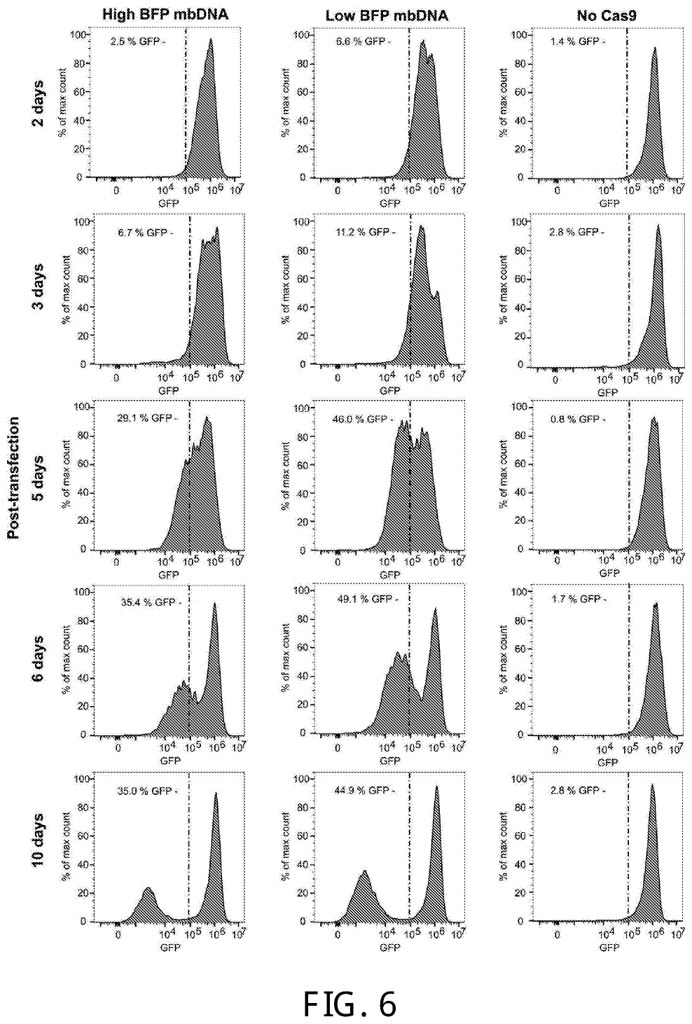

HEK293T-EGFP cells transfected with Cas9+sgRNA plasmid and BFP mbDNA (delivery vector) showed a gradual reduction of EGFP over the course of 6 days following transfection. On day 6, between 35% and 50% of cells had stopped expressing EGFP (FIG. 6) demonstrating the gene targeting ability of Cas9 in our system. Cells transfected with a high amount (450 ng) of BFP delivery vector lost EGFP at a slower rate compared to cells with a low amount (45 ng) of BFP delivery vector. This reduced rate of gene editing can be explained by the slightly reduced efficiency of the high mbDNA transfections (approximately 70% of cells with high BFP/control vector showed Scarlet-I expression 48 h post-transfection, compared to approximately 80% for low/no vector). No further loss of EGFP was evident past day 6, as shown by data from day 10 post-transfection (HEK293T-EGFP cells expressing Cas9 are losing EGFP). Histograms showing GFP signal as percentage of maximum counted events as measured by flow cytometry in cells transfected with either high (450 ng) or low (45 ng) BFP delivery vector at indicated timepoints post-transfection. Dotted line indicates threshold for GFP-positive signal.

Percentage of GFP-negative events in each sample are quoted (FIG. 6), suggesting that no Cas9 activity is detectable beyond day 6. In contrast, cells not expressing Cas9 exhibited no reduction in EGFP, validating that EGFP loss is Cas9-mediated.

As used “mbDNA” is the vector, and it is indicated whether this is includes the delivery of BFP or is the control (no BFP).

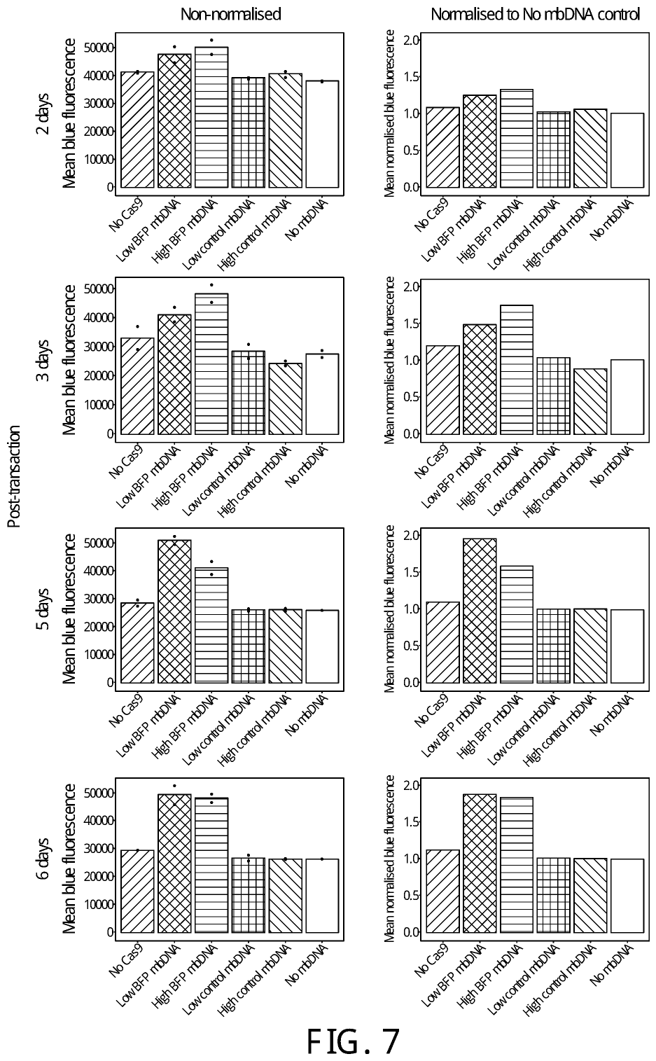

Successful homology-directed recombination (HDR) gene editing events were identified by measuring blue fluorescence protein (BFP) intensity in lysates from cells on days 2-6 following introduction of BFP delivery vector and control vector. As soon as day 2 post-transfection, cells with BFP, but not control vector, showed a 1.3-fold increase in BFP signal relative to the no vector control (FIG. 7). On day 5, BFP intensity from cells with high BFP vector was 2-fold higher compared to control and by day 6, the same relative levels of BFP were detectable in both cells with low and high BFP vector (FIG. 7). Importantly, increased BFP signal cannot be explained by interference from the EGFP signal, as cells without Cas9 (No Cas9)—and therefore expressing more functional EGFP—have a lower BFP signal compared to Cas9-transfected cells (FIG. 7).

Altogether, our data demonstrates that BFP delivery vector according to the present invention is cleavable by Cas9 in vivo and can release a viable transgene that can be used as an HDR template in Cas9-mediated gene editing.

1. A nucleic acid delivery vector comprising a circular single stranded polynucleotide said vector comprising:
(a) a duplex formed from a first section and a third section of said polynucleotide, said sections including sequences which are complementary;
(b) a loop formed from a second section, said section separating the first and third sections;
wherein said duplex includes a recognition sequence for a targeted nuclease.

2. The nucleic acid delivery vector of claim 1 wherein said vector delivers a linear single stranded nucleic acid, wherein said single stranded nucleic acid is present within the second section.

3. The nucleic acid delivery vector of claim 2 wherein the linear single stranded nucleic acid may be any one or more of: a nucleic acid enzyme, an aptamer, a donor template, an mRNA, a functional RNA, or an antisense nucleic acid.

4. The nucleic acid delivery vector of claim 2 or 3 wherein the linear single stranded nucleic acid has a free 5′ and 3′ end once released from the delivery vector.

5. The nucleic acid delivery vector of claim 1 wherein said vector is a closed circular polynucleotide, optionally a closed DNA or closed RNA.

6. The nucleic acid delivery vector of any previous claim wherein said nuclease is a guided nuclease, optionally a nuclease associated with gene editing, preferably Cas9.

7. The nucleic acid delivery vector of any one of claims 1 to 5 wherein the nuclease binds to the recognition sequence without a guide.

8. The nucleic acid delivery vector of any previous claim wherein said vector is for use in a cell.

9. A method of providing a linear single stranded nucleic acid to a cell, comprising the use of a delivery vector as claimed in any one of claims 1 to 8.

10. A method of providing a linear single stranded donor template to a cell for genome editing, comprising the use of a delivery vector as described in any one of claims 1 to 8, preferably wherein said linear single stranded nucleic acid is a donor template.

11. A method as claimed in claim 10 wherein said nuclease is a guided nuclease, optionally Cas9 or a variant thereof.
